(19) United States
(12) Patent Application Publication
(10) Pub. No.: US 2023/0390086 A1
(43) Pub. Date: Dec. 7, 2023

(54) Volitional Walking Controller

(71) Applicant: UNIVERSITY OF UTAH RESEARCH FOUNDATION, Salt Lake City, UT (US)

(72) Inventors: Joel MENDEZ, Salt Lake City, UT (US); Tommaso LENZI, Salt Lake City, UT (US)

(21) Appl. No.: 18/033,052

(22) PCT Filed: Oct. 21, 2021
(86) PCT No.: PCT/US2021/056093

§ 371 Date: Apr. 20, 2023

Related U.S. Application Data
(60) Provisional application No. 63/104,022, filed on Oct. 22, 2020.

(51) Int. Cl. A61F 2/70; A61F 2/64; A61F 2/66

(52) U.S. Cl. CPC A61F 2/70; A61F 2002/7625; A61F 2/6607; A61F 2/64

(57) ABSTRACT

A powered prosthesis for providing volitional control of knee flexion during swing is configured to (i) determine that a swing phase has initiated, (ii) obtain a thigh angle based on the sensor data associated with a residual limb of a user, (iii) based on a time elapsed since initiation of the swing phase, and based on the thigh angle, determine a desired maximum knee flexion angle, (iv) during the swing phase, continuously update the desired maximum knee flexion angle using subsequent measurements of thigh angle and time elapsed since initiation of the swing phase, and (v) output a signal configured to cause actuation of the knee joint based on the desired maximum knee flexion angle.

### CROSS-REFERENCE TO RELATED APPLICATIONS

This application claims priority to and the benefit of U.S. Provisional Patent Application Ser. No. 63/104,022, filed Oct. 22, 2020 and titled “Volitional Walking Controller”, the entirety of which is incorporated herein by this reference.

### STATEMENT REGARDING FEDERALLY SPONSORED RESEARCH

This invention was made with government support under grant nos. HD098154 awarded by the National Institutes of Health and 1925371 awarded by the National Science Foundation. The government has certain rights in this invention.

### BACKGROUND

### Technical Field

This disclosure generally relates to powered prostheses that provide volitional control of knee joint actuation. In particular, this disclosure relates to powered prostheses for above-knee amputations that provide control over knee joint actuation based at least partly on a determined thigh angle of the user's residual limb, through the use of continuous minimum-jerk planning to re-program desired prosthesis trajectory at multiple time points during the swing phase.

### Related Technology

Most available knee prostheses are energetically passive devices with limited ability to reproduce the behavior of the healthy biological knee. In the simplest devices, the biomechanical behavior of the healthy knee joint is approximated by friction elements or hydraulic dampers. More advanced knee prostheses use a microcontroller to change the knee damping withing the gait cycle. These microprocessor-controlled knees allow for variable cadence while improving stance stability and reducing metabolic cost of walking compared to friction and hydraulic knees. Despite these improvements, ambulation is slower, less stable, and less efficient for individuals with above-knee amputation than able-bodied individuals. Moreover, negotiating common environmental barriers such as curbs, stairs, or uneven surfaces requires unnatural, destabilizing compensatory movements such as residual hip circumduction and plantar flexion of the sound ankle (i.e., vaulting) to compensate for the missing prosthesis knee flexion.

In contrast to passive prostheses, powered prostheses can actively regulate joint movements using battery-powered servomotors. To accomplish this goal, powered prostheses typically use controllers that aim to replicate the behavior of the healthy leg across different ambulation activities. One common control method consists of dividing the gait cycle into segments that are characteristic of the nominal gait pattern, such as stance and swing. In swing, the trajectory of the powered prosthetic joint is often determined by joint impedance parameters such as stiffness, damping, and equilibrium point, which are tuned by the experimenter to imitate the nominal knee joint trajectory. To accomplish this goal, swing must be divided into two segments with different impedance parameters. Furthermore, tuning of impedance parameters is necessary to change the swing timing as necessary to walk at different speeds. Despite their robustness, impedance-based controllers have limited adaptability, making it hard to adapt the swing trajectory to the user's needs.

Position-based controllers have been more recently proposed to simplify the tuning procedure and provide more flexibility over the powered prosthesis behavior. Rather than using a set of impedance parameters, position-based controllers define the whole swing trajectory either as a continuous function of time or residual-limb movements. Using this approach, the desired position trajectory can be conveniently extracted from the analysis of able-bodied biomechanics, which avoids the need for manual tuning. The desired trajectory can then be stored in look-up tables, encoded using parametric functions, or obtained online using minimum jerk programming. This control approach has been used successfully for different locomotion tasks such as variable-speed walking, variable inclines, and stair climbing. However, this control approach lacks the ability to adapt the prosthesis trajectory outside of the nominal gait pattern. Thus, available position-based controllers are not suitable to traverse environmental barriers such as curbs and uneven surfaces.

Classification-based controllers have been proposed to achieve ambulation over different terrains. The basic idea is to use separate controllers for different environmental conditions, which must be detected by an online classifier. The detection of environmental obstacles or constraints can rely on a combination of mechanical sensors, electromyography, sonomyography, lasers, or computer vision. These classification-based controllers have been originally developed for detecting ramps and stairs, and, more recently, have been used for obstacle detection. However, open challenges remain in obtaining an accuracy that is conducive to use in the community as well as in the training of the classification algorithms. Therefore, the clinical viability of classification-based controllers is uncertain.

Accordingly, improved prothesis technologies are necessary to address the needs of individuals with an above-knee amputation.

### SUMMARY

The present disclosure describes prostheses devices configured to adapt the prosthesis trajectory (e.g., knee and/or ankle joint angle over time) continuously during swing phase to enable enhanced volitional control of the prosthesis and to enable enhanced ability to traverse environmental barriers. Prostheses described herein represent an improvement over conventional powered prostheses, many of which operate by planning the desired prosthesis trajectory at the beginning of swing and keeping it constant throughout the swing duration. Prostheses described herein also represent an improvement over conventional powered prostheses that operate using a classification-based approach. That is, prostheses described herein modulate the swing trajectory according to movements of the user's residual limb without requiring an explicit classification of the environment.

The prostheses described herein beneficially enable users with above-knee amputations to volitionally control foot clearance, enabling such users to better navigate environmental barriers such as by enabling more controlled crossing over obstacles of different sizes.

In one embodiment, a powered prosthesis configured to provide volitional control of knee flexion during swing comprises a knee joint; one or more sensors for determining a thigh angle of a residual limb to which the prosthesis is attached; and a controller communicatively coupled to the knee joint and the one or more sensors. The controller includes one or more processors and one or more hardware storage devices having stored thereon computer-executable instructions. The controller is configured to determine that the swing phase has initiated; receive the thigh angle from the one or more sensors; and based on the time elapsed since initiation of the swing phase, and based on the received thigh angle, determine a desired maximum knee flexion angle. The controller then updates, at multiple time points during swing, the desired maximum knee flexion angle using subsequent measurements of thigh angle and time elapsed since initiation of the swing phase.

The controller can be further configured to determine a first swing state and a second swing state within the swing phase. The first swing state functions to control knee flexion and the second swing state functions to slow and end knee flexion, if necessary, and control knee extension. The controller transitions from the first swing state to the second swing state upon determining that the thigh angle has passed a thigh angle threshold or upon determining that the time elapsed since initiation of the swing phase has exceeded a time threshold. The thigh angle threshold may be variable. For example, the thigh angle threshold may vary as a function of the desired maximum knee flexion angle as determined and updated over time by the controller during swing.

The controller can be configured to determine the desired maximum knee flexion angle using the integral of the thigh angle over a time period from the initiation of the swing phase to the present duration of the swing phase. The controller can be configured to determine that the swing phase has initiated upon determining a ground reaction force (GRF) that is lower than a stance-to-swing threshold, the stance-to-swing threshold being proportional to a body weight of the user. The controller can also be configured to determine a transition from the swing phase to a first stance state upon determining a GRF that is higher than a swing-to-stance threshold, the swing-to-stance threshold being proportional to a body weight of the user. The controller can also be configured to determine a transition between a first stance state (i.e., a default standing state) and a second stance state (i.e., an energy-injection state) by determining that the ankle joint exceeds a dorsiflexion threshold and has positive plantarflexion velocity.

The controller can be configured to determine a desired knee joint position, velocity, and acceleration using a minimum jerk engine. The minimum jerk engine receives as inputs the desired maximum knee flexion angle, and the time remaining until desired duration of a first swing state, and outputs updated desired knee joint position, velocity, and acceleration to enable determination and updating of desired knee torque for the knee joint.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 illustrates an example powered prosthesis for an above-knee amputee, the prosthesis including powered knee and ankle joints;

FIG. 2 illustrates a state machine that may be utilized by the controller of the prosthesis, showing respective transitions between stance states and swing states;

FIG. 3 schematically illustrates components of the example controller;

FIGS. 4 through 6 illustrates various simulations of the prosthesis at different time of transition from a first swing state to a second swing state;

FIG. 7 illustrates an example flow diagram depicting acts associated with providing volitional control of a powered prosthesis, in accordance with implementations of the present disclosure;

FIGS. 8 through 12 illustrate various kinematics of a powered prosthesis resulting from subject testing;

FIG. 13 illustrates comparisons between subjects wearing a prosthesis and able-bodied subjects through six different walking tests, illustrating that the kinematics of the subjects wearing the prosthesis implementing the disclosed controller were substantially similar to the kinematics of the able-bodied control subjects; and

FIGS. 14 through 16 illustrate the kinematics of a prosthesis as compared to the kinematics of a conventional passive prosthesis and the kinematics of a biological leg, showing that the prosthesis implementing the disclosed controller more closely resembles the kinematics of the biological leg than does the conventional passive prosthesis.

### DETAILED DESCRIPTION

### Introduction

Crossing over obstacles with conventional passive prostheses requires individuals with above-knee amputations to circumduct the ipsilateral hip (i.e., prosthesis side) and plantarflex the contralateral ankle (i.e., sound side vaulting) to compensate for the lack of knee flexion. Powered prostheses have the potential to address this issue by directly controlling the knee flexion during swing. However, available powered prothesis controllers cannot automatically adapt the swing trajectory as necessary to traverse environmental barriers such as crossing over obstacles.

Classification-based controllers aim to address this issue by switching between different pre-planned swing trajectories that are appropriate to deal with different ambulation tasks and environmental barriers. However, this classification approach requires the prosthesis controller to detect the desired ambulation task or environmental barrier online before it is negotiated by the prosthesis user. This classification is typically performed using machine learning, which requires extensive training data sets. Furthermore, close to 100% online accuracy is necessary for classification-based controllers to work properly, as both a false positive and false negative may result in dangerous behavior of the powered prosthesis, potentially causing the prosthesis user to fall.

Conventional approaches have not provided the prosthesis user with volitional control of maximum knee flexion or foot clearance. Here, rather than classifying the environmental barrier and switching between different, pre-planned swing trajectories, the inventive prostheses continuously modulate the trajectory of the powered prosthesis based on the movements of the user's residual limb so that environmental barriers can be negotiated. This approach beneficially enables individuals with an above-knee amputation to ambulate at different speeds while seamlessly crossing over obstacles.

Biomechanics studies on able-bodied subjects show that crossing over an obstacle requires increasing both hip and knee flexion to shorten the limb and create clearance between the foot and the obstacle. Accordingly, continuous adaptation of the prosthesis knee flexion in swing can beneficially change foot clearance as required to cross over obstacles. The modulation of foot clearance using the controller described herein is obtained through two factors also observed in able-bodied individuals. The first factor is the increase of maximum knee flexion. The second factor is the time shift in the movements of the user's residual limb and the prosthetic knee, such as for the same thigh angle, the prosthesis knee flexion angle increases when crossing over obstacles.

### Example Powered Knee and Ankle Prosthesis

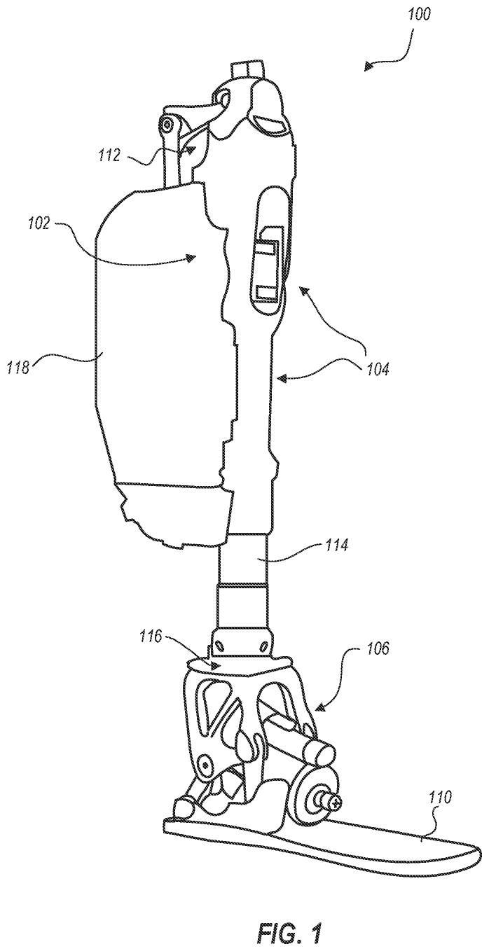

Systems, methods, and techniques related to adaptive volitional control of powered prostheses, in accordance with the present disclosure, may be implemented utilizing various types of knee and ankle prostheses. FIG. 1 illustrates a perspective view of an example powered knee and ankle prosthesis 100 that may be implemented in conjunction with the principles disclosed herein related to adaptive volitional control of powered prostheses. One will appreciate, in view of the present disclosure, that the particular components and/or features of the powered knee and ankle prosthesis 100 of FIG. 1 do not limit the applicability of the disclosed principles to other types of powered knee and ankle prostheses that include additional or alternative components.

The example powered knee and ankle prosthesis 100 of FIG. 1 comprises a self-contained, battery-operated, powered knee and ankle prosthesis that can generate biologically appropriate torque and power during ambulation. The powered knee and ankle prosthesis 100 of FIG. 1 may be configured and/or adjustable to fit users associated with various body sizes. For example, the powered knee and ankle prosthesis 100 may be sized to fit the 50th percentile male leg profile. The powered knee and ankle prosthesis 100 may comprise any suitable weight, such as within a range of about 1.5 kg to about 8 kg (e.g., about 2.5 kg with the battery and protective covers included).

The example powered knee and ankle prosthesis 100 of FIG. 1 comprises an ankle-foot module 106. The ankle-foot module 106 may utilize a compact, lightweight powered polycentric design, which may be contained within a commercially available foot shell. The powered polycentric mechanism of the ankle-foot module 106 may be connected/connectable to custom carbon-fiber feet 110 of different sizes to accommodate different subjects.

The example powered knee and ankle prosthesis 100 of FIG. 1 further comprises a knee module 104. The knee module 104 may utilize an active variable transmission 112 (AVT 112) to optimize the effective transmission ratio and leg dynamics for different locomotion tasks. In addition, the knee module 104 may contain/comprise a control unit and battery 102 and/or motor drivers for both the knee joint and the ankle joint. The knee module 104 and the ankle-foot module 106 may connect with a pylon 114 (e.g., a standard 30-mm pylon), which may allow for height and intra-extra rotation adjustments. In some instances, a pyramid adapter 116 is implemented at the top of the ankle-foot module 106 to estimate the ground reaction force and torque.

The AVT 112 of the example powered knee and ankle prosthesis 100 of FIG. 1 utilizes a DC motor (e.g., a Maxon Motor EC 13, 18 V, 12 W) connected to a 4.1:1 planetary gear, which drives the nut on a bigger, non-backdrivable leadscrew (e.g., M4x1.25, single start) through a 1:1 spur gear transmission. In the example powered knee and ankle prosthesis 100 of FIG. 1, two thrust bearings and two ball bearings are used to support the leadscrew's axial and radial loads. In addition, the lead screw of the AVT 112 can be supported by two parallel guides realized by slotted cranks with dry bushings (e.g., IGUS® Iglidur® L280, static friction coefficient 0.23, dynamic friction coefficient 0.08-0.23). The slotted crank defines the range of motion of the AVT 112 (e.g., a range of motion within a range of about 20 mm to about 45 mm). The overall structural safety factor of the example powered knee and ankle prosthesis 100 represented in FIG. 1 is 2.5. An incremental encoder (e.g., RLS, RM08) is, in some instances, located on the spur gear to measure the position of the AVT 220. This sensor (i.e., the incremental encoder), together with a four-quadrant motor driver (e.g., Maxon Motor ESCON module 24/2) may enable feedback control of the position of the AVT 112 in both driving and braking operations. As noted above, other configurations are within the scope of the present disclosure.

The primary actuator of the example powered knee and ankle prosthesis 100 represented in FIG. 1 is a rotary-to-linear system comprising a brushless DC motor (e.g., Maxon Motor EC-4pole 22, 24 V, 120 W), a roller screw (e.g., Rollvis®, pitch diameter 4.5 mm, lead 2 mm, static-dynamic load ratings 7.2-7.8 kN, efficiency 90%), and a timing-belt transmission (e.g., 48:18 teeth ratio). The roller screw nut is supported by a linear guide (e.g., Helix Linear Technologies, HMR9ML, basic load/moment ratings 3880 N/12.4 Nm). The main motor can be located inside of an aluminium frame (e.g., 7075 T6-SN) which may also operate as a heatsink. A force-torque sensor is, in some instances, embedded in the pylon 114 to detect contact with the ground. Furthermore, in some implementations, a 9-DOF IMU (MPU9250, Invsense) is included to sense the movements and the orientation of the leg in space.

Covers 118 (e.g., 3D printed covers) may be utilized to house the control unit and battery 102. The control unit and battery 102 may comprise a Li-Ion battery (e.g., 2500 mAh, 6S) and/or an onboard system-on-module (SOM) (e.g., myRIO 1900, National Instruments, 100 g without covers). The SOM can run all custom control algorithms in real time, interfacing with the sensors and servo drivers for the AVT 112 and the primary motor (e.g., Elmo, Gold Twitter G-TWI 30/60SE, 35 g). The SOM can be connected through Wi-Fi to a host computer, smartphone, and/or other device for data monitoring and/or controller tuning.

Experimental results (discussed in more detail hereinafter) were obtained by implementing an adaptive volitional controller with a powered knee and ankle prosthesis 100 that includes the features/components discussed with reference to FIG. 1. However, as noted previously, the principles discussed herein related to shared volitional control of powered prostheses are not limited to the particular components/features of the powered knee and ankle prosthesis 100 discussed above with reference to FIG. 1.

### Powered Prosthesis with Adaptive Volitional Controller

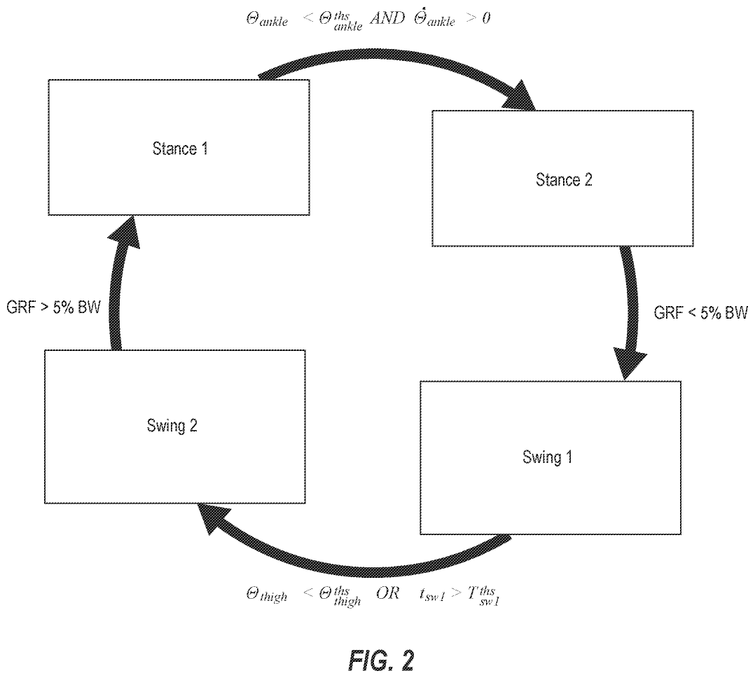

FIG. 2 illustrates a state machine that may be utilized by the controller of the prosthesis, showing respective transitions between stance states and swing states. In Stance 1, the prosthesis absorbs the impact with the ground, storing and dissipating energy as necessary. The prosthesis transitions to Stance 2 when the ankle position is greater than a specific dorsiflexion angle and the ankle velocity is positive (i.e., the ankle is plantarflexing). When the instrumented pyramid detects the GRF being lower than 5% of the user's body weight, the controller transitions to Swing 1. In Swing 1, the disclosed controller adapts the prosthesis trajectory based on the movements of the residual limb. If the residual limb exceeds a variable threshold, the prosthesis switches to Swing 2, where a minimum jerk trajectory is generated to prepare the foot for the subsequent heel-strike.

When the user is standing still, the prosthesis controller is in Stance 1. If the ankle joint exceeds a dorsiflexion threshold (θₐₙₖₗₑ<θₐₙₖₗₑ^{ths}) and has positive plantarflexion velocity (\dot{θ}ₐₙₖₗₑ>0), the system transitions to Stance 2, which is an energy-injection state. From Stance 2, the prosthesis transitions to Swing 1 when a force sensor integrated with the prosthesis (e.g., an instrumented pyramid adapter) detects a GRF lower than some proportion of the of the user's body weight (e.g., 5% of the user's body weight).

In Swing 1, the knee joint flexes to increase foot clearance. In this state, the knee joint trajectory is modulated by the controller to continuously change the desired maximum knee flexion. From Swing 1, the system transitions to Swing 2 when the orientation of the user's residual limb crosses a position threshold (θ_{thigh}<θ_{thigh}^{ths}) or if duration of Swing 1 exceeds a time threshold (t_{sw1}>T_{sw1}^{ths}). In Swing 2, a knee extension trajectory is programmed enabling timely placement of the prosthetic foot in preparation for the subsequent heel strike. Finally, the prosthesis transitions from Swing 2 to Stance 1 when the force sensor detects the GRF higher than some proportion of the user's body weight (e.g., 5% of the user's body weight).

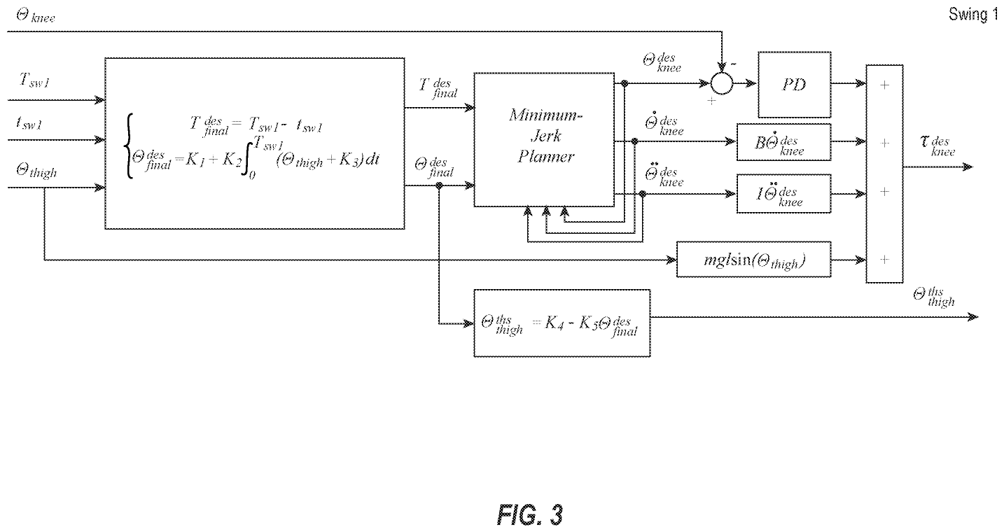

FIG. 3 illustrates components of the adaptive volitional controller. According to the block diagram of FIG. 3, the desired knee angle at the end of Swing 1 (θ_{final}^{des}) is continuously updated based on the orientation of the user's residual limb with respect to gravity (θ_{thigh}) using an integral function (Equation (1)). Based on the desired knee angle (θ_{final}^{des}) and the remaining time in Swing 1 (T_{final}^{des}), the minimum jerk planner computes the desired knee angle trajectory (θₖₙₑₑ^{des}, \dot{θ}ₖₙₑ^{des}, \ddot{θ}ₖₙₑₑ^{des}), which is then passed to a closed-loop position controller including a proportional-derivative regulator (PD) and feedforward compensators for viscosity and inertia. In addition, the threshold for transitioning between Swing 1 and Swing 2 (θ_{thigh}^{ths}) is updated based on the desired knee angle (θ_{final}^{des}) and passed on to the finite-state machine.

As shown, the desired maximum knee flexion angle (θ_{final}^{des}) in Swing 1 is determined by the integral of the residual limb orientation with respect to gravity (θ_{thigh}), computed from the start to the end of Swing 1 (or to the time duration of Swing 1 up to the present time of measurement), according to Equation (1):

θ_{final}^{des}(t)=K₁+K₂∫₀^{T}^{_{sw1}}(θ_{thigh}(t)+K₃)dt

In the example presented in FIG. 3, three (optional) parameters affect the desired maximum knee flexion angle (θ_{final}^{des}) The first parameter is a constant (e.g., K₁=55) that determines the desired angle at the start of the swing movement, when the integral output is zero. The second parameter is a gain (e.g., K₂=2) that affects the sensitivity of the integral. The third parameter (e.g., K₃=20) is a constant added to the thigh angle (θ_{thigh}), which provides a bias for the output of the integral. Preferred ranges of these three parameters, as described herein, have been determined from both the analysis of able-bodied biomechanics and pilot testing with individuals with amputations. The same “default” parameters may be used for all users, or alternatively, customized parameters may be determined through use.

According to Equation (1), the desired maximum knee flexion angle (θ_{final}^{des}) increases when the residual limb is positioned farther back (i.e., when θ_{thigh} is larger), or when it is moved forward slowly during Swing 1 (right after toe-off). Thus, the prosthesis knee swing trajectory can be modulated by the user through movement of the residual limb, enabling variable foot clearance.

Given a desired maximum knee flexion angle (θ_{final}^{des}), the swing trajectory is continuously optimized using minimum jerk. As shown, the minimum-jerk planner (also referred to herein as minimum-jerk engine) takes as input the desired maximum knee flexion angle (θ_{final}^{des}) and the desired movement duration (T_{final}^{des}), which is computed in Swing 1 by subtracting the current swing time (t_{sw1}(t)) from the desired Swing 1 duration (T_{sw1}), as shown by Equation (2):

T_{final}^{des}(t)=T_{sw1}−t_{sw1}(t)

Based on these inputs and on the previously determined desired position, velocity, and acceleration, the minimum jerk planner updates the desired swing trajectory by computing the desired angle, velocity, and acceleration of the knee joint. The desired angle, velocity, and acceleration are then passed to a mixed feedforward/feedback regulator that determines the desired torque at the knee joint level. A new trajectory can branch off from the swing trajectory originally programmed at toe-off if the desired final position or the desired swing duration change. Thus, with continuous minimum-jerk planning, the prosthesis can smoothly change the swing trajectory while it is being performed regardless of the current angle, velocity, and acceleration of the prosthesis joint.

Although the desired maximum knee flexion (θ_{final}^{des}) is computed through the integral of the residual limb orientation (θ_{thigh}), the actual peak of knee flexion depends on the position of the knee at the transition between Swing 1 and Swing 2. The finite-state machine transitions from Swing 1 to Swing 2 when the thigh angle (θ_{thigh}) exceeds a threshold (θ_{thigh}^{ths}). However, this threshold is not fixed, but rather varies as a function of the desired peak knee flexion (θ_{final}^{des}) as defined by Equation (3):

θ_{thigh}^{ths}(t)=K₄−K₅θ_{final}^{des}(t)

Where, by way of non-limiting example, K₄ equals 17.5 and K₅ equals 0.5. These constants may take other values as determined through empirical testing and/or individualized customization of a prosthesis.

Based on Equation (3), the thigh threshold at the transition between Swing 1 and Swing 2 increases proportionally to the desired peak knee flexion (θ_{final}^{des}). Thus, the transition between Swing 1 and Swing 2 can happen at different points within Swing. The desired duration of Swing 1 (T_{sw1}) can be set as a constant (e.g., at 0.4 s) that can be determined empirically, for example.

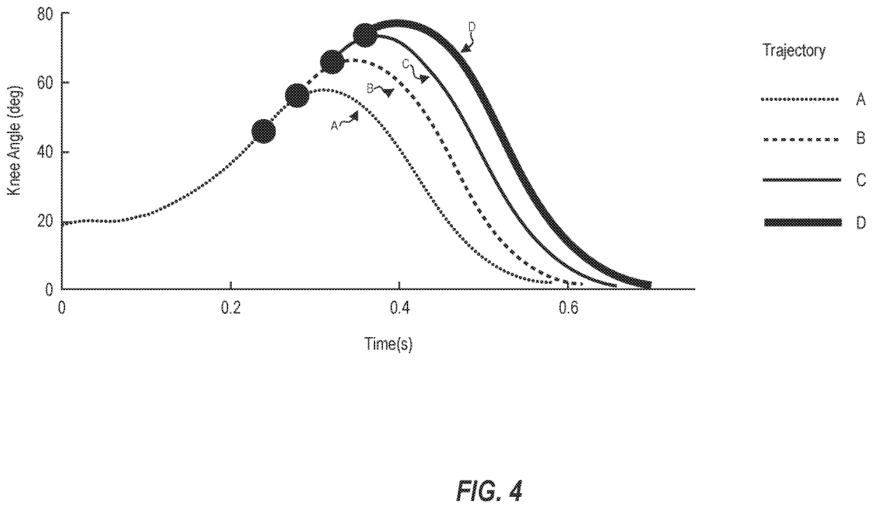

In Swing 2, the prosthesis uses a minimum jerk controller to ensure timely placement of the foot in preparation for the subsequent heel strike. As shown by the simulation results of FIG. 4, the system can transition to Swing 2 with different knee positions and velocities. As a result, the desired trajectory in Swing 2 may allow for both knee flexion and extension, effectively modulating the maximum knee flexion achieved by the powered prosthesis in Swing. This controller behavior contrasts with previous controllers, where Swing 2 is used for knee extension only.

In stance, the controller enforces physiological torque-angle curves extracted from able-bodied individuals walking at different speeds. Thus, the knee and ankle torque profiles are adapted online based on the respective joint positions and an overall estimate of the current walking speed. Different from impedance-based controllers, this stance controller does not necessarily require user-specific or speed-specific tuning, although the body weight of the user is preferably inserted in the controller. Other stance controllers may alternatively be utilized as any initial angle, speed, and acceleration can be handled by the minimum-jerk swing controller.

A longer stride and a larger knee flexion are shown to produce a higher clearance in able-bodied individuals. As a result, the controller continuously modulates the maximum knee flexion in swing depending on how far back the residual limb (i.e., thigh angle) is positioned and how fast the user moves it forward during the flexion part of swing. Moreover, it was designed to adjusts Swing 1 duration, as able-bodied individuals typically wait to start extension at a higher thigh angle whenever a higher clearance is desired. This heuristic adaptation of maximum knee flexion and Swing 1 duration can be combined with minimum jerk programming to obtain a smooth behavior of the leg that qualitatively matches the behavior of the healthy leg.

Previous prostheses using a minimum-jerk approach program the trajectory at toe-off and keep it constant through the whole swing duration. Thus, the swing trajectory cannot be modified as necessary to cross over obstacles. In contrast, the present controller updates the swing trajectory at multiple instances within the swing phase.

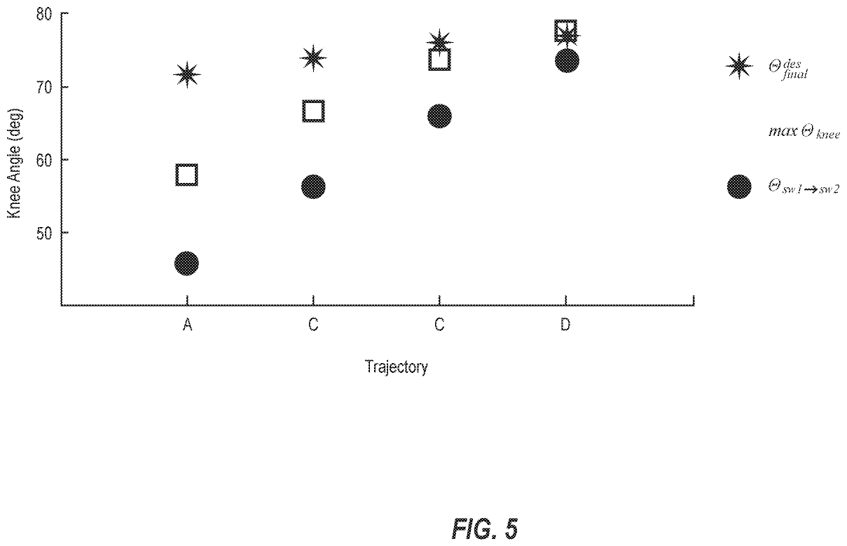

The behavior of the controller is demonstrated by the simulations in FIGS. 4-6 Error! Reference source not found. In FIG. 4, swing trajectories (A-D) for four possible transition points between Swing 1 and Swing 2 are shown using solid lines. The transition points are shown using circle markers. In FIG. 5, the desired knee maximum in Swing 1 (θ_{final}^{des}), actual knee maximum (max θₖₙₑₑ), and knee angle at the transition between Swing 1 and Swing 2 (θ_{sw1→sw2}) for four simulated trajectories (A-D) are shown.

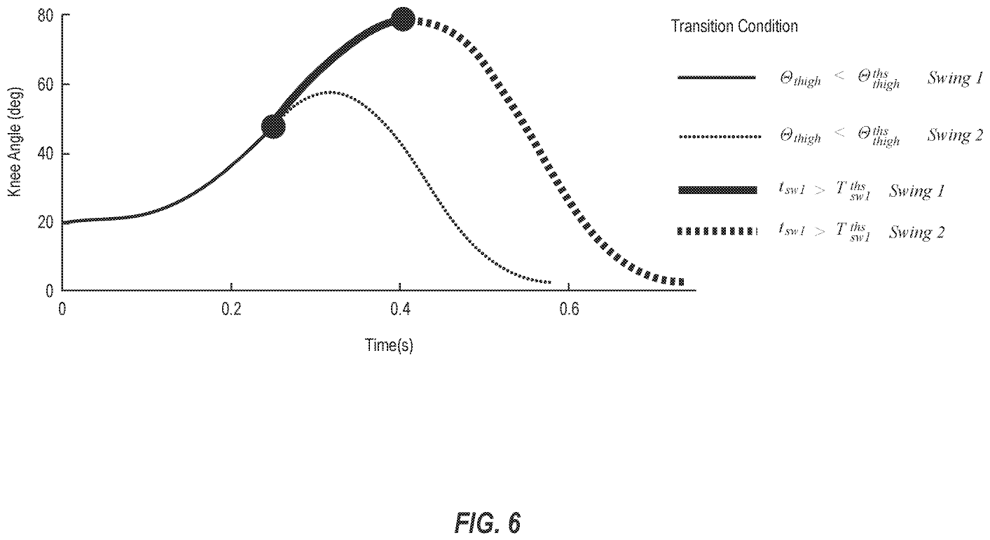

In FIG. 6, swing trajectories for two transition points determined by the thigh position condition (θ_{thigh}<θ_{thigh}^{ths}, light grey) and the timeout condition (t_{sw1}>T_{sw1}^{ths}, dark grey). The solid line shows the desired position trajectory in Swing 1; the dashed line shows the desired position trajectory in Swing 2.

Varying the transition points between Swing 1 and Swing 2 (circle markers in FIG. 4) results in a modulation of the swing trajectory (solid lines). A later transition point results in a higher knee flexion angle (max θₖₙₑₑ) and a longer overall duration of Swing (see FIGS. 4 and 5). The control system transitions between Swing 1 and Swing 2 even if the desired peak of knee flexion in Swing 1 (θ_{final}^{des}) is not achieved. Thus, the maximum knee flexion (θ_{final}^{des}) in Swing 1 does not necessarily match the actual maximum of the desired knee swing trajectory (max θₖₙₑₑ), which can be achieved in Swing 2 (see FIG. 5).

However, if the transition between Swing 1 and Swing 2 is due to the timeout conditions (circle marker in FIG. 6), then the desired peak knee flexion (θ_{final}^{des}) matches the actual peak of the knee trajectory (solid line in Error! Reference source not found. FIG. 6). As a result, the transition between Swing 1 and Swing 2 can happen at a different time within the gait cycle, different knee flexion angles, and different orientations of the user's residual limb. Thus, the variable transition condition between Swing 1 to Swing 2 enables the powered knee to perform different swing trajectories, allowing for volitional variations of foot clearance while still maximizing smoothness.

### Example Methods

The following discussion now refers to a number of methods and method acts that may be performed in accordance with the present disclosure. Although the method acts are discussed in a certain order and illustrated in a flow chart as occurring in a particular order, no particular ordering is required unless specifically stated, or required because an act is dependent on another act being completed prior to the act being performed. One will appreciate that certain embodiments of the present disclosure may omit one or more of the acts described herein.

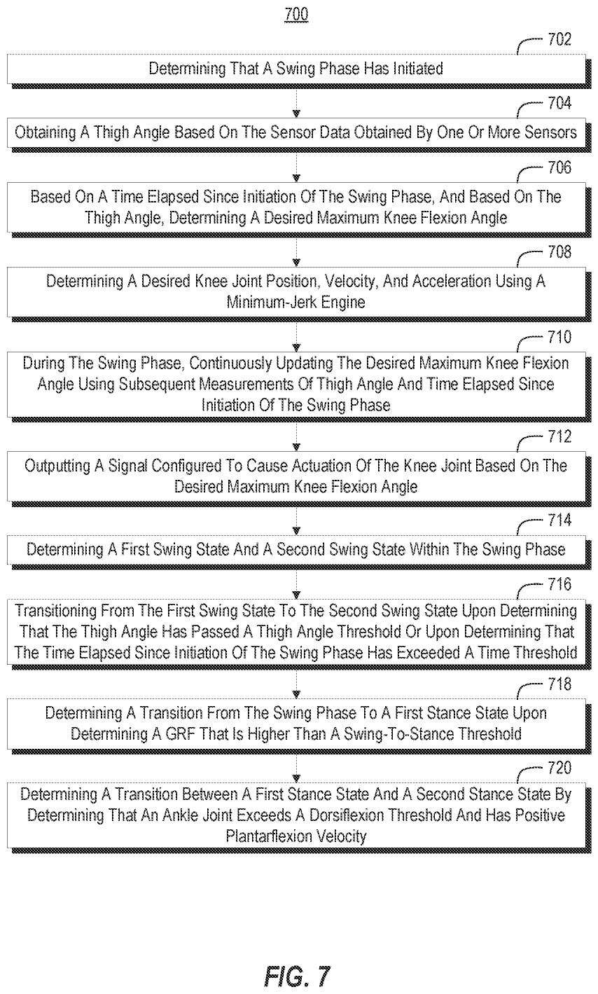

FIG. 7 illustrates an example flow diagram 700 depicting acts associated with providing volitional control of prosthesis joint movement, in accordance with the present disclosure. The acts depicted in flow diagram 700 may be performed utilizing various hardware elements discussed hereinabove, such as controllers (e.g., of control unit and battery 102), sensor(s), motors, etc. A controller may comprise one or more processing devices and may comprise or access one or more hardware storage devices to facilitate execution of stored instructions to carry out one or more of the acts/functions described herein.

Act 702 of flow diagram 700 includes determining that a swing phase has initiated. In some instances, sensor data from a force sensor for measuring ground reaction force (GRF) is used to determine that the swing phase has initiated upon detecting a GRF that is lower than a stance-to-swing threshold. The stance-to-swing threshold may be proportional to a body weight of a user.

Act 704 of flow diagram 700 includes obtaining a thigh angle based on the sensor data obtained by one or more sensors.

Act 706 of flow diagram 700 includes, based on a time elapsed since initiation of the swing phase, and based on the thigh angle, determining a desired maximum knee flexion angle. In some instances, the desired maximum knee flexion angle is determined using an integral of the thigh angle over a time period from the initiation of the swing phase to a present duration of the swing phase. For example, the desired maximum knee flexion angle may be determined according to:

θ_{final}^{des}(t)=K₁+K₂∫₀^{T}^{_{sw1}}(θ_{thigh}(t)+K₃)dt

where (t) is the time elapsed since initiation of the swing phase, θ_{final}^{des}(t) is the desired maximum knee flexion angle, θ_{thigh}(t) is the thigh angle at time (t), T_{sw1} is a desired duration of a first swing state, and K₁ , K₂, and K₃ are optional constants. In some implementations, K₁ is within a range of about 40 to about 70, or within a range of about to about 60, or about 55. In some implementations, K₂ is within a range of about 1.1 to about 3, or within a range of about 1.5 to about 2.5, or about 2. In some implementations, K₃ is within a range of about 5 to about 35, or within a range of about 10 to about 30, or within a range of about 15 to about 25. In some implementations, T_{sw1} is within a range of about 0.25 s to about 0.65 s, or within a range of about 0.35 s to about 0.45 s, or about 0.4 s.

Act 708 of flow diagram 700 includes determining a desired knee joint position, velocity, and acceleration using a minimum jerk engine. The minimum-jerk engine may receive as inputs the desired maximum knee flexion angle, and a desired movement duration. The minimum-jerk engine may output updated desired knee joint position, velocity, and acceleration.

Act 710 of flow diagram 700 includes, during the swing phase, continuously updating the desired maximum knee flexion angle using subsequent measurements of thigh angle and time elapsed since initiation of the swing phase. The minimum jerk engine referred to above in act 708 may accordingly update the desired knee joint position, velocity, and acceleration based on the updated desired maximum knee flexion angle. Therefore, knee flexion of the knee joint during the swing phase may be controlled without explicit classification of an environment.

Act 712 of flow diagram 700 includes outputting a signal configured to cause actuation of the knee joint based on the desired maximum knee flexion angle. For example, the signal may be configured to cause actuation of the knee joint in accordance with the desired knee joint position, velocity, and acceleration (e.g., determined utilizing the minimum-jerk engine).

Act 714 of flow diagram 700 includes determining a first swing state and a second swing state within the swing phase. The first swing state may be associated with control of knee flexion, and the second swing state may be associated with control of knee extension and/or, if necessary, slowing and ending knee flexion. In some instances, the minimum-jerk engine is used to control knee joint movement during the second swing state.

Act 716 of flow diagram 700 includes transitioning from the first swing state to the second swing state upon determining that the thigh angle has passed a thigh angle threshold or upon determining that the time elapsed since initiation of the swing phase has exceeded a time threshold. In some implementations, the thigh angle threshold is variable, such as by varying as a function of the desired maximum knee flexion angle. For example, the thigh angle threshold may be determined according to:

θ_{thigh}^{ths}(t)=K₄−K₅θ_{final}^{des}(t)

where θ_{final}^{ths}(t) is the thigh angle threshold, θ_{final}^{des}(t) is the desired maximum knee flexion angle, K₄ is a constant, and K₅ is an optional constant. In some implementations, K₄ is within a range of about 10 to about 25, or about 17.5. In some implementations, K₅ is within a range of about 0.25 to about 0.75, or about 0.5.

Act 718 of flow diagram 700 includes determining a transition from the swing phase to a first stance state upon determining a GRF that is higher than a swing-to-stance threshold. In some instances, the swing-to-stance threshold is proportional to a body weight of a user. For example, the swing-to-stance threshold may be within a range of about 3% to about 10% of the body weight of the user, or about 5% of the body weight of the user.

Act 720 of flow diagram 700 includes determining a transition between a first stance state and a second stance state by determining that an ankle joint exceeds a dorsiflexion threshold and has positive plantarflexion velocity. The second stance state may comprise an energy-injection state.

### Example Results

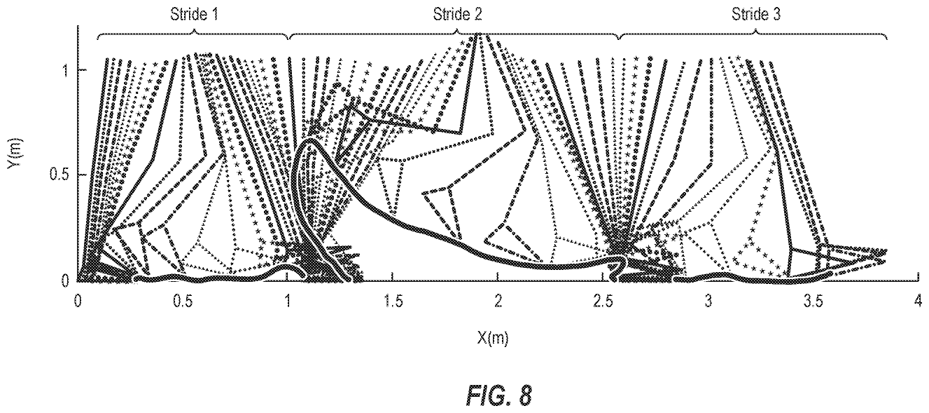

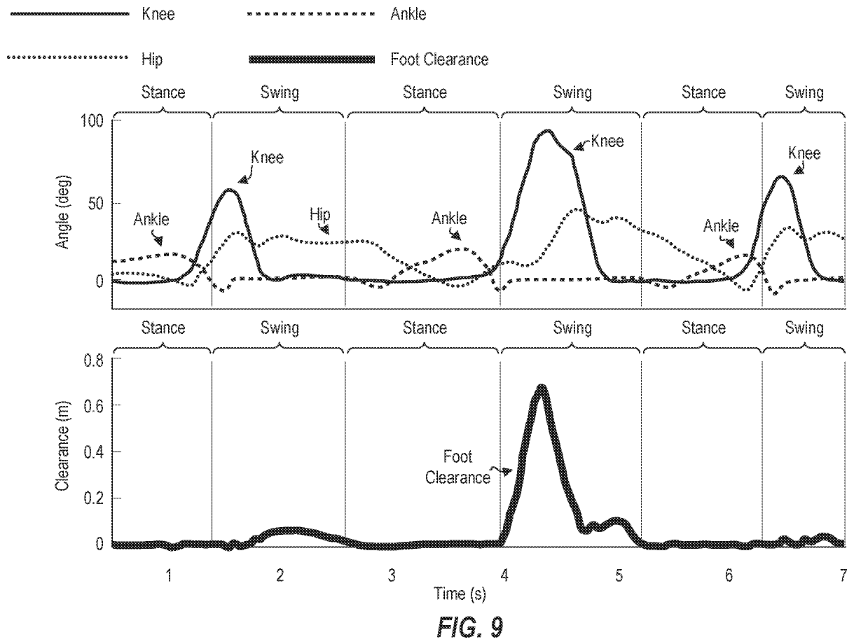

The functionality of the volitional walking controller was measured by observing its performance during a series of tests. In the first test, subjects walked back and forth at their self-selected speed on a 4-m walkway including starting and stopping, while an obstacle was placed in the middle of the walkway. Three different obstacles sizes were used in different trials and are denoted as small (10 cm×80 cm×6 cm), medium (15 cm×80 cm×10 cm), and large (30 cm×80 cm×20 cm). A representative test with one subject with an above-knee amputation crossing over the medium-size obstacle is shown in FIGS. 8 and 9.

As can be seen, the subject performs three consecutive strides with the obstacle being crossed in the second stride with the sound side first. The gait pattern changes considerably when the subject crosses over the obstacle. Specifically, the range of motion of the hip joint increases from 34° and 39° for the first and last stride, respectively, to 51° for the obstacle crossing stride. As a result, a 46% longer stride is taken when crossing the obstacle (x axis, FIG. 8). The maximum knee flexion is 56°, 94°, and 64° for the first, second, and third stride, respectively. Thus, when crossing over the obstacle, the prosthesis achieves a 68% larger maximum knee flexion. As expected, the ankle kinematics is not visibly affected by the presence of the obstacle. The peak of foot clearance increases from 0.06 m and 0.02 m in the first and third stride, respectively, to 0.67 m for the third stride. Thus, when crossing over the obstacle, the subject holds back the residual limb longer, causing the unified controller to increase the maximum knee flexion angle and, consequently, the foot clearance.

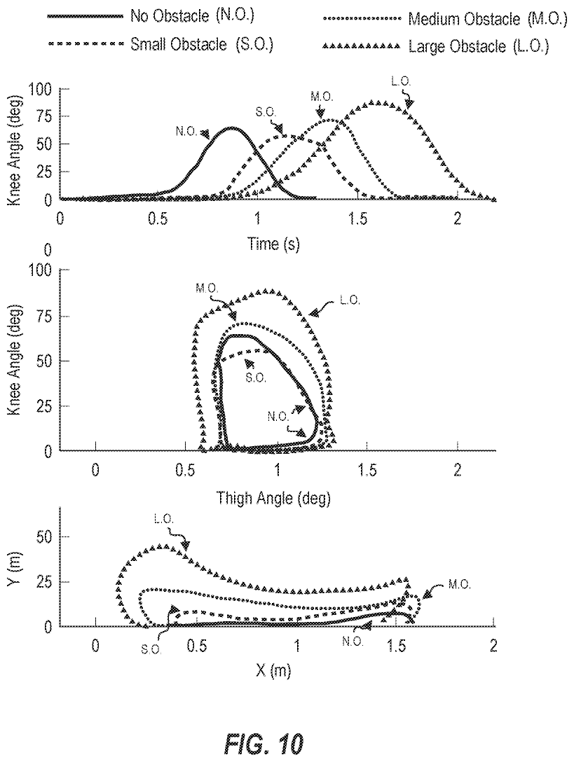

By analyzing the gait kinematics during the level-ground test with and without obstacles, assess the ability of a subject to voluntarily change foot clearance can be assessed. The analysis of the powered knee kinematics for a representative subject shows that the stride duration is longer in the presence of an obstacle and that it increases with the obstacle size. Similarly, the maximum knee flexion increases with the obstacle size. Moreover, the inversion of the movement between knee flexion and extension (see FIG. 10) is generally slower when crossing over the obstacle, showing a rather flat region for the small and large obstacle.

The phase analysis (FIG. 10) shows that the timing between the residual limb motion (i.e., thigh angle, x-axis) and the powered knee angle (i.e., y-axis) changes when the subject crosses over different obstacles. Specifically, during Swing 1 the knee angle is generally larger when crossing over the obstacle than when no obstacle is present. Finally, larger foot clearance is observed when crossing obstacles of increasing sizes (FIG. 10). Thus, with the disclosed swing controller, the subject is able to change the powered knee kinematics and foot clearance as necessary to walk over ground and cross over obstacles of different sizes at his preferred walking speed.

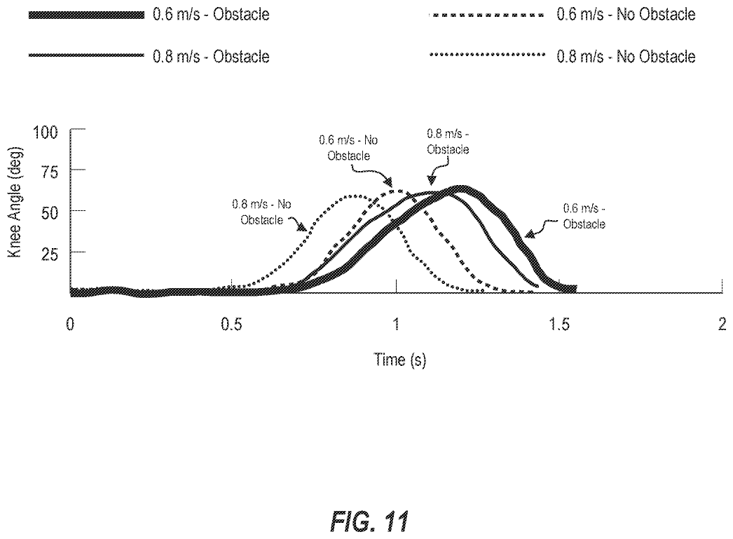

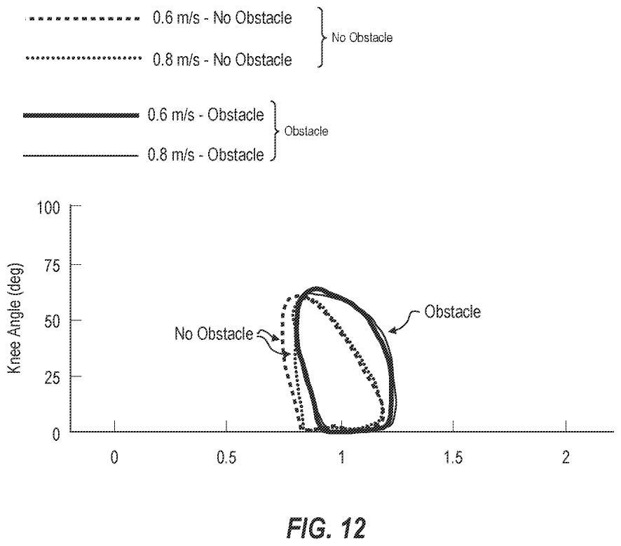

The performance of the controller under continuous walking is demonstrated by the subjects walking on a treadmill at two different speeds (i.e., 0.6 m/s, 0.8 m/s) while an experimenter manually drops 6-cm tall obstacles on the belt in the path of the powered prosthesis. The analysis of the knee kinematics (FIGS. 11 and 12) for one representative subject shows that the stride duration is generally longer for the slower walking speed than the faster walking speed. In addition, for the same walking speed, the stride duration is longer when the subject crosses the obstacle than when no obstacle is encountered. At the slower speed (0.6 m/s), the maximum knee flexion is 60.4±2.1° without an obstacle, and 64.0±2.3° with an obstacle. At the faster speed (0.8 m/s), the maximum knee flexion is 60.0±0.9° when no obstacle is encountered, and 61.1±6.1° when an obstacle is encountered.

The phase analysis (i.e., knee angle vs. the thigh angle) shows that timing of the knee and thigh movements is altered when an obstacle is crossed (FIG. 12). In Swing 1, the knee angle tends to be larger when an obstacle is crossed. On the other hand, the phase plots are not visibly affected by the treadmill speed. In turn, the altered timing of the knee and thigh movements produce a larger foot clearance (i.e., +261% for the slow treadmill speed and +206% for the fast treadmill speed) when crossing the obstacle. Thus, with the controller, the subject is able to walk at the two different speeds imposed by the treadmill with and without crossing over obstacles.

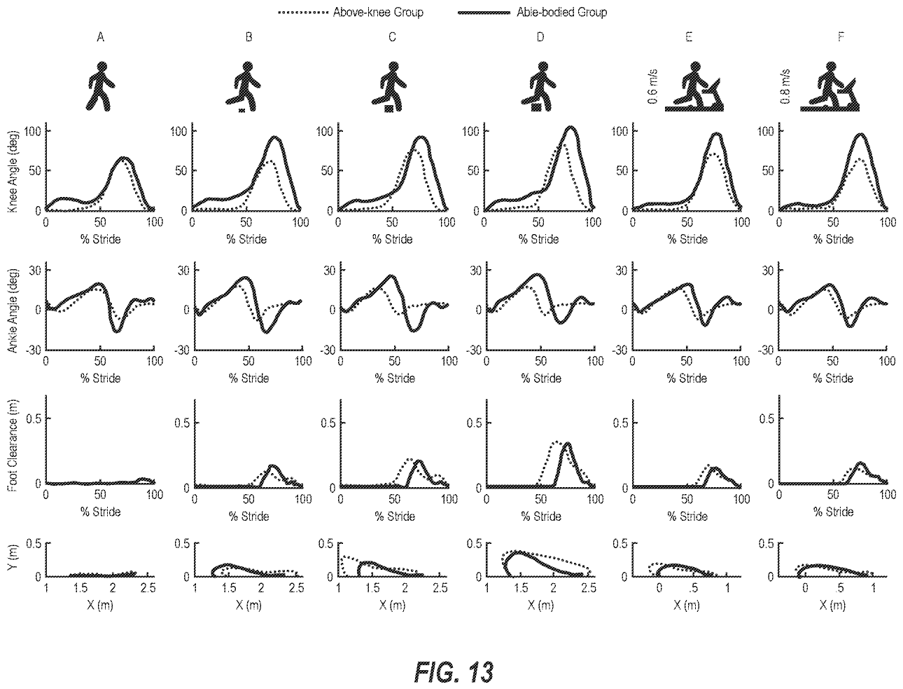

As shown by FIG. 13, the comparison between the kinematics of able-bodied and above-knee subjects performing the same cross-over obstacles tests provides further insights into the behavior of the disclosed volitional controller. The knee angle trajectories observed in the two groups (i.e. above-knee; able-bodied) during the test with no obstacle (A) match closely in swing (60-100% of the gait cycle). Overall, the maximum knee flexion angle tends to be greater for able-bodied subjects than for subjects with an above-knee amputation using the disclosed swing controller. In the level-ground walking tests (A-D), there is a difference of 26.3°, 16.7°, and 15.7° for the small obstacle, medium obstacle, and large obstacle respectively. However, when no obstacle is encountered, both groups reach similar maximum knee flexion with a 0.4° difference being recorded.

The difference in maximum knee flexion is larger for the treadmill tests, with it reaching 20.8° and 25.7° for the 0.6 m/s and 0.8 m/s speed respectively. By focusing on the level-ground tests with obstacles (B-D, FIG. 13), It is evident that the maximum knee flexion tends to occur later in the gait cycle for able-bodied subjects than for subjects with an above-knee amputation. On average, the above-knee group reaches their maximum knee flexion at 68% of stride whenever they cross any type of obstacle on level-ground. The able-bodied subjects on the other hand, reach their maximum knee flexion at 77% of stride. When no obstacle is encountered, the above-knee amputee group reach their maximum flexion at 68% of stride, while the able-bodied subjects reach maximum flexion at 72% of stride.

The timing of the knee flexion is more similar during the treadmill tests, where subjects with an above-knee amputation reach their maximum flexion at an average of 75% of stride and able-bodied subjects reach their maximum flexion at an average of 77% of stride. Moreover, in able-bodied individuals, heel strike happens as soon as the knee is fully extended, whereas subjects with an above-knee amputation tend to delay heel strike. During level-ground walking with no obstacle (A, FIG. 13), knee extension is completed at 92% of stride among the above-knee amputation group. On the other hand, for the small, medium, and large obstacle test (B-D, FIG. 13), knee extension ends at 91%, 85% and 95% of stride respectively.

The heel strike delay is less pronounced during the treadmill test (E-F, FIG. 13), where extension ends at 98% of stride and 99% of stride for the slower and faster walking speeds, respectively. The two groups show similar average trajectories (solid lines), noticeably with the same time delay for the knee and ankle kinematics. The cartesian representation of the foot clearance, shows that individuals with amputations tend to have higher foot clearance at the beginning of swing, when the foot raises from the ground. In contrast, a similar foot clearance is observed in the two groups at the point where the obstacle is located. Thus, able-bodied subjects and subjects with an above-knee amputation show different joint kinematics but similar foot clearance when walking and crossing over obstacles.

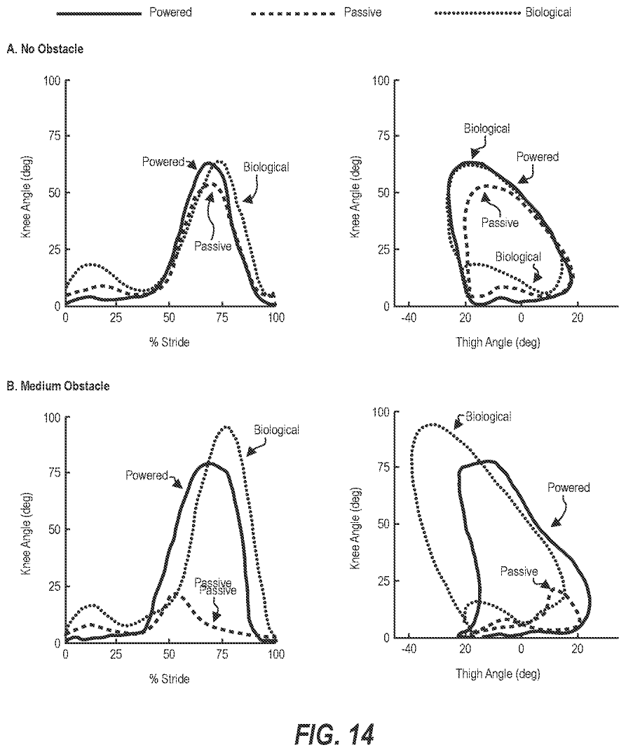

As shown by FIG. 14, the analysis of obstacle crossing with conventional passive prostheses provides a reference to assess the volitional controller of the present disclosure. The kinematic of the passive prosthesis, the powered prosthesis, and the able-bodied biological leg are fairly similar when no obstacle is encountered (A, FIG. 14). When the obstacle is encountered (B, FIG. 14), the knee flexion increases for the powered prosthesis and the biological leg, although the observed knee flexion is smaller with the powered prosthesis than the biological leg. Similar to able-bodied subjects, individuals with an above-knee amputation can change the timing between knee flexion and thigh position (i.e., residual limb) with the powered prosthesis (B, FIG. 14). In contrast, the knee flexion angle decreases when an obstacle is encountered with the passive prosthesis (B, FIG. 14). Due to the lack of knee flexion, the passive prosthesis hits the obstacles for all tests and subjects. In contrast, the foot clearance with the powered prosthesis volitional controller is sufficient to cross over obstacles up to 20-cm tall.

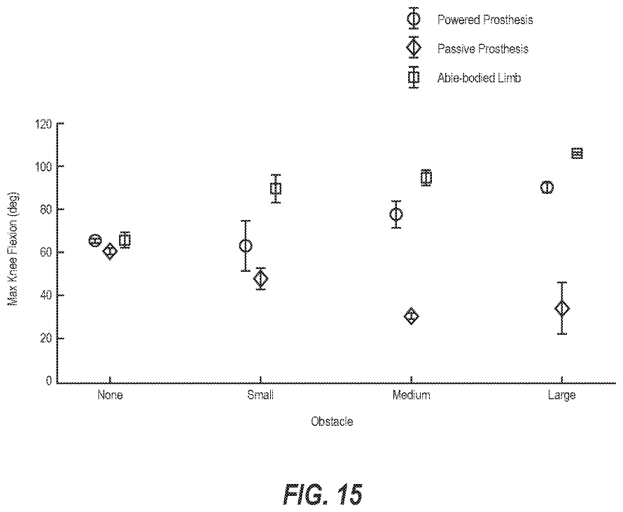

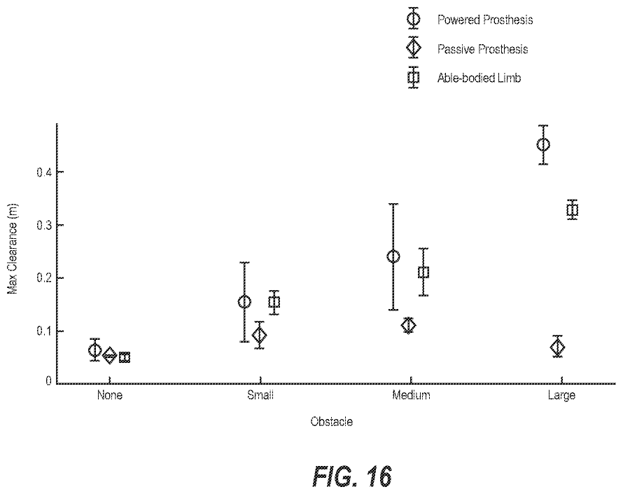

FIGS. 15 and 16 show the behavior of the disclosed controller by comparing the maximum knee flexion and maximum foot clearance between different groups (i.e., able bodied, above-knee) and obstacle conditions (i.e., small, medium, large). Similar to able-bodied subjects, above-knee subjects using the powered prosthesis increase both the maximum knee flexion (FIG. 15) and the foot clearance (FIG. 16) with the size of the obstacle. However, when an obstacle is crossed, the maximum knee flexion is larger for able-bodied subjects than for above-knee subjects using the powered prosthesis. Interestingly, above-knee subjects show smaller knee flexion but greater foot clearance than able-bodied subject (FIG. 16). Furthermore, the variability of the maximum knee flexion decreases with the size of the obstacle for both the powered prosthesis and able-bodied tests (FIG. 15). The maximum knee flexion and foot clearance of the passive prosthesis are much lower than the powered prosthesis and biological leg. Moreover, the foot clearance with the passive prosthesis does not change with obstacle size. Thus, able-bodied subjects and individuals with amputations using the powered prosthesis show similar trends of maximum knee angle and foot clearance as a function of the obstacle size although an offset between the two groups is observed.

### Additional Example Aspects

Embodiments of the present disclosure may include, but are not necessarily limited to, features recited in the following clauses:
Clause 1: a powered prosthesis configured to provide volitional control of knee flexion during swing, the prosthesis comprising: a knee joint; one or more sensors configuring for obtaining sensor data associated with a residual limb to which the powered prosthesis is attached; and a controller the one or more sensors, the controller including one or more processors and one or more hardware storage devices storing computer-executable instructions that are executable by the one or more processors to configure the controller to: determine that a swing phase has initiated; obtain a thigh angle based on the sensor data obtained by the one or more sensors; based on a time elapsed since initiation of the swing phase, and based on the thigh angle, determine a desired maximum knee flexion angle; during the swing phase, continuously update the desired maximum knee flexion angle using subsequent measurements of thigh angle and time elapsed since initiation of the swing phase; and output a signal configured to cause actuation of the knee joint based on the desired maximum knee flexion angle.
Clause 2: the powered prosthesis of Clause 1, wherein knee flexion of the knee joint during the swing phase is controlled without explicit classification of the environment.
Clause 3: the powered prosthesis of Clause 1 or Clause 2, wherein the controller is further configured to: determine a first swing state and a second swing state within the swing phase, wherein the first swing state controls knee flexion and the second swing state controls knee extension and/or, if necessary, slows and ends knee flexion; and transition from the first swing state to the second swing state upon determining that the thigh angle has passed a thigh angle threshold or upon determining that the time elapsed since initiation of the swing phase has exceeded a time threshold.
Clause 4: the powered prosthesis of Clause 3, wherein the thigh angle threshold is variable.
Clause 5: the powered prosthesis of Clause 4, wherein the thigh angle threshold varies as a function of the desired maximum knee flexion angle.
Clause 6: the powered prosthesis of any one of Clauses 1 through 5, wherein the desired maximum knee flexion angle is determined using an integral of the thigh angle over a time period from the initiation of the swing phase to a present duration of the swing phase.
Clause 7: the powered prosthesis of Clause 6, wherein the desired maximum knee flexion angle is determined according to:

θ_{final}^{des}(t)=K₁+K₂∫₀^{T}^{_{sw1}}(θ_{thigh}(t)+K₃)dt

wherein (t) is the time elapsed since initiation of the swing phase, θ_{final}^{des}(t) is the desired maximum knee flexion angle, θ_{thigh}(t) is the thigh angle at time (t), T_{sw1} is a desired duration of a first swing state, and K₁ , K₂, and K₃ are optional constants.
Clause 8: the powered prosthesis of Clause 7, wherein K₁ is within a range of about 40 to about 70, or within a range of about 50 to about 60, or about 55.
Clause 9: the powered prosthesis of Clause 7 or Clause 8, wherein K₂ is within a range of about 1.1 to about 3, or within a range of about 1.5 to about 2.5, or about 2.
Clause 10: the powered prosthesis of any one of Clauses 7 through 9, wherein K₃ is within a range of about 5 to about 35, or within a range of about 10 to about 30, or within a range of about 15 to about 25.
Clause 11: the powered prosthesis of any one of Clauses 7 through 10, wherein T_{sw1} is within a range of about 0.25 s to about 0.65 s, or within a range of about 0.35 s to about 0.45 s, or about 0.4 s.
Clause 12: the powered prosthesis of any one of Clauses 4 through 11, wherein the thigh angle threshold is determined according to:

θ_{thigh}^{ths}(t)=K₄−K₅θ_{final}^{des}(t)

wherein θ_{thigh}^{ths}(t) is the thigh threshold, θ_{final}^{des}(t) is the desired maximum knee flexion angle, K₄ is a constant, and K₅ is an optional constant.
Clause 13: the powered prosthesis of Clause 12, wherein K₄ is within a range of about 10 to about 25, or about 17.5.
Clause 14: the powered prosthesis of Clause 12 or Clause 13, wherein K₅ is within a range of about 0.25 to about 0.75, or about 0.5.
Clause 15: the powered prosthesis of any one of Clauses 1 through 14, further comprising a force sensor for measuring ground reaction force (GRF), and wherein the controller is further configured to determine that the swing phase has initiated upon determining a GRF that is lower than a stance-to-swing threshold, the stance-to-swing threshold being proportional to a body weight of the user.
Clause 16: the powered prosthesis of Clause 15, wherein the stance-to-swing threshold is within a range of about 3% to about 10% of the body weight of the user, or about 5% of the body weight of the user.
Clause 17: the powered prosthesis of any one of Clauses 1 through 16, wherein the controller is further configured to determine a transition from the swing phase to a first stance state upon determining a GRF that is higher than a swing-to-stance threshold, the swing-to-stance threshold being proportional to a body weight of the user.
Clause 18: the powered prosthesis of Clause 17, wherein the swing-to-stance threshold is within a range of about 3% to about 10% of the body weight of the user, or about 5% of the body weight of the user.
Clause 19: the powered prosthesis of any one of Clauses 1 through 18, further comprising an ankle joint, wherein the controller is configured to determine a transition between a first stance state and a second stance state by determining that the ankle joint exceeds a dorsiflexion threshold and has positive plantarflexion velocity.
Clause 20: the powered prosthesis of Clause 19, wherein the second stance state is an energy-injection state.
Clause 21: the powered prosthesis of any one of Clauses 1 through 20, wherein the controller is further configured to determine a desired knee joint position, velocity, and acceleration using a minimum jerk engine, wherein the minimum jerk engine receives as inputs the desired maximum knee flexion angle, and a desired movement duration, and wherein the minimum jerk engine outputs updated desired knee joint position, velocity, and acceleration.
Clause 22: the powered prosthesis of any one of Clauses 1 through 21, wherein the controller is configured to determine a first swing state and a second swing state within the swing phase, wherein the first swing state functions to control knee flexion and the second swing state functions to slow and end knee flexion, if necessary, and control knee extension, wherein the controller uses a minimum-jerk engine to control knee joint movement during the second swing state.
Clause 23: a method for providing volitional control of knee flexion during swing, comprising: determining that a swing phase has initiated; obtaining a thigh angle based on sensor data obtained by one or more sensors, the sensor data being associated with a residual limb to which a powered prosthesis is attached; based on a time elapsed since initiation of the swing phase, and based on the thigh angle, determining a desired maximum knee flexion angle; and during the swing phase, continuously updating the desired maximum knee flexion angle using subsequent measurements of thigh angle and time elapsed since initiation of the swing phase.
Clause 24: one or more hardware storage devices storing instructions that are executable by one or more processors of a controller to configure the controller to provide volitional control of knee flexion during swing by configuring the controller to: determine that a swing phase has initiated; obtain a thigh angle based on the sensor data obtained by the one or more sensors; based on a time elapsed since initiation of the swing phase, and based on the thigh angle, determine a desired maximum knee flexion angle; and during the swing phase, continuously update the desired maximum knee flexion angle using subsequent measurements of thigh angle and time elapsed since initiation of the swing phase.

### Additional Terms & Definitions

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, or less than 1% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may include properties, features (e.g., components, members, elements, parts, and/or portions) described in other embodiments described herein. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

What is claimed is:

1. A powered prosthesis configured to provide volitional control of knee flexion during swing, the prosthesis comprising:
a knee joint;
one or more sensors configuring for obtaining sensor data associated with a residual limb to which the powered prosthesis is attached; and
a controller the one or more sensors, the controller including one or more processors and one or more hardware storage devices storing computer-executable instructions that are executable by the one or more processors to configure the controller to:
determine that a swing phase has initiated;
obtain a thigh angle based on the sensor data obtained by the one or more sensors;
based on a time elapsed since initiation of the swing phase, and based on the thigh angle, determine a desired maximum knee flexion angle;
during the swing phase, continuously update the desired maximum knee flexion angle using subsequent measurements of thigh angle and time elapsed since initiation of the swing phase; and
output a signal configured to cause actuation of the knee joint based on the desired maximum knee flexion angle.

2. The powered prosthesis of claim 1, wherein knee flexion of the knee joint during the swing phase is controlled without explicit classification of an environment.

3. The powered prosthesis of claim 1, wherein the controller is further configured to:
determine a first swing state and a second swing state within the swing phase, wherein the first swing state controls knee flexion and the second swing state controls knee extension and/or, if necessary, slows and ends knee flexion; and
transition from the first swing state to the second swing state upon determining that the thigh angle has passed a thigh angle threshold or upon determining that the time elapsed since initiation of the swing phase has exceeded a time threshold.

4. The powered prosthesis of claim 3, wherein the thigh angle threshold is variable.

5. The powered prosthesis of claim 4, wherein the thigh angle threshold varies as a function of the desired maximum knee flexion angle.

6. The powered prosthesis of claim 1, wherein the desired maximum knee flexion angle is determined using an integral of the thigh angle over a time period from the initiation of the swing phase to a present duration of the swing phase.

7. The powered prosthesis of claim 6, wherein the desired maximum knee flexion angle is determined according to:
θ_{final}^{des}(t)=K₁+K₂∫₀^{T}^{_{sw1}}(θ_{thigh}(t)+K₃)dt
wherein (t) is the time elapsed since initiation of the swing phase, θ_{final}^{des}(t) is the desired maximum knee flexion angle, θ_{thigh}(t) is the thigh angle at time (t), T_{sw1} is a desired duration of a first swing state, and K₁, K₂, and K₃ are optional constants.

8. The powered prosthesis of claim 7, wherein K₁ is within a range of about 40 to about 70, or within a range of about 50 to about 60, or about 55.

9. The powered prosthesis of claim 7, wherein K₂ is within a range of about 1.1 to about 3, or within a range of about 1.5 to about 2.5, or about 2.

10. The powered prosthesis of claim 7, wherein K₃ is within a range of about 5 to about 35, or within a range of about 10 to about 30, or within a range of about 15 to about 25.

11. The powered prosthesis of claim 7, wherein T_{sw1} is within a range of about 0.25 s to about 0.65 s, or within a range of about 0.35 s to about 0.45 s, or about 0.4 s.

12. The powered prosthesis of claim 3, wherein the thigh angle threshold is determined according to:
θ_{thigh}^{ths}(t)=K₄−K₅θ_{final}^{des}(t)
wherein θ_{final}^{ths}(t) is the thigh angle threshold, θ_{final}^{des}(t) the desired maximum knee flexion angle, K₄ is a constant, and K₅ is an optional constant.

13. The powered prosthesis of claim 12, wherein K₄ is within a range of about 10 to about 25, or about 17.5.

14. The powered prosthesis of claim 12, wherein K₅ is within a range of about 0.25 to about 0.75, or about 0.5.

15. The powered prosthesis of claim 1, further comprising a force sensor for measuring ground reaction force (GRF), and wherein the controller is further configured to determine that the swing phase has initiated upon determining a GRF that is lower than a stance-to-swing threshold, the stance-to-swing threshold being proportional to a body weight of a user.

16. The powered prosthesis of claim 15, wherein the stance-to-swing threshold is within a range of about 3% to about 10% of the body weight of the user, or about 5% of the body weight of the user.

17. The powered prosthesis of claim 1, wherein the controller is further configured to determine a transition from the swing phase to a first stance state upon determining a GRF that is higher than a swing-to-stance threshold, the swing-to-stance threshold being proportional to a body weight of a user.

18. The powered prosthesis of claim 17, wherein the swing-to-stance threshold is within a range of about 3% to about 10% of the body weight of the user, or about 5% of the body weight of the user.

19. The powered prosthesis of claim 1, further comprising an ankle joint, wherein the controller is configured to determine a transition between a first stance state and a second stance state by determining that the ankle joint exceeds a dorsiflexion threshold and has positive plantarflexion velocity.

20. The powered prosthesis of claim 19, wherein the second stance state is an energy-injection state.

21. The powered prosthesis of claim 1,
wherein the controller is further configured to determine a desired knee joint position, velocity, and acceleration using a minimum jerk engine,
wherein the minimum-jerk engine receives as inputs the desired maximum knee flexion angle, and a desired movement duration, and
wherein the minimum-jerk engine outputs updated desired knee joint position, velocity, and acceleration.

22. The powered prosthesis of claim 1, wherein the controller is configured to determine a first swing state and a second swing state within the swing phase, wherein the first swing state functions to control knee flexion and the second swing state functions to slow and end knee flexion, if necessary, and control knee extension, wherein the controller uses a minimum-jerk engine to control knee joint movement during the second swing state.

23. A method for providing volitional control of knee flexion during swing, comprising:
determining that a swing phase has initiated;
obtaining a thigh angle based on sensor data obtained by one or more sensors, the sensor data being associated with a residual limb to which a powered prosthesis is attached;
based on a time elapsed since initiation of the swing phase, and based on the thigh angle, determining a desired maximum knee flexion angle; and
during the swing phase, continuously updating the desired maximum knee flexion angle using subsequent measurements of thigh angle and time elapsed since initiation of the swing phase.

24. One or more hardware storage devices storing instructions that are executable by one or more processors of a controller to configure the controller to provide volitional control of knee flexion during swing by configuring the controller to:
obtain a thigh angle based on sensor data obtained by one or more sensors, the sensor data being associated with a residual limb to which a powered prosthesis is attached;
based on a time elapsed since initiation of the swing phase, and based on the thigh angle, determine a desired maximum knee flexion angle; and
during the swing phase, continuously update the desired maximum knee flexion angle using subsequent measurements of thigh angle and time elapsed since initiation of the swing phase.
